(19) United States
(12) Patent Application Publication
(10) Pub. No.: US 2006/0024376 A1
(43) Pub. Date: Feb. 2, 2006

(54) Methods and compositions for reducing toxicity associated with leflunomide treatment

(75) Inventor: James W. Williams, Oak park, IL (US)

Correspondence Address: MICHAEL BEST & FRIEDRICH, LLP, ONE SOUTH PINCKNEY STREET, P O BOX 1806, MADISON, WI, 53701, US

(73) Assignee: The University of Chicago, Chicago, IL (US)

(21) Appl. No.: 10/903,681

(22) Filed: Jul. 30, 2004

(51) Int. Cl. A61K 31/513; A61K 31/505; A61K 9/14

(52) U.S. Cl. 424/489; 514/256; 514/269

(57) ABSTRACT

The invention relates to methods and compositions useful in alleviating or reducing toxicity associated with leflunomide administration without reducing its bioactivity, e.g., without reducing its immunosuppressive activity, that is, utilizing a bioavailable pyrimidine compound to ameloriate the toxic effects caused by leflunomide compounds.

Leflunomide is an isoxazole derivative which has shown therapeutic potential in a diverse array of disease processes and conditions, e.g., as an antiinflammatory agent, an immunosuppressive agent, an anticancer agent and an antiviral agent.

Leflunomide is currently approved in the United States for use in the treatment of rheumatoid arthritis to reduce joint inflammation. It is marketed under the trademark ARAVA®.

U.S. Pat. Nos. 5,624,946 and 5,688,824, incorporated herein by reference in their entirety, report that leflunomide has been used experimentally as an immunosuppressive agent in the treatment and prevention of chronic rejection in xenograft and allograft transplant recipients, both alone and in combination with other immunosuppressive agents.

In addition to data suggesting its value in treating, preventing and reversing acute and chronic rejection, U.S. Patent Application U.S. 2003/0114597, incorporated herein by reference in its entirety, reports that leflunomide has been shown to inhibit viruses of the Herpesviridae family in vitro.

U.S. Pat. No. 4,965,276 describes the use of leflunomide to treat chronic graft versus host disease and other autoimmune diseases such as systemic lupus erythematosus (SLE). Leflunomide has also been shown to exhibit antineoplastic activity against certain tumors (Xu X et al., Biochem. Pharmacol. 1999; 58: 1405) and may act by inhibiting tumor neoangiogenesis (Waldman W J et al., Transplantation 2001; 72:1578).

Despite the reported therapeutic benefits of leflunomide in the treatment and prevention of these disease processes, it has also been noted that administration of leflunomide may produce dose-limiting toxicity. Toxicity associated with high doses of leflunomide include anemia, diarrhea, and pathological changes of the small intestine and liver. In a study of the anti-cancer effects of leflunomide (inhibition of the oncogene product PDGF and PDGFr) observable beneficial effects were reported but the doses required for these effects produced unacceptable incidence of side effects, including severe weight loss, anorexia and anemia. (Ko, Yoo-Joung, et al. Clinical Cancer Research, 2001;7: 800-805)

Recently, it has been suggested that uridine therapy reduces the toxicity of leflunomide without significantly impairing the control of allograft rejection afforded by leflunomide. The utilization of exogenous uridine occurs through the pyrimidine salvage pathway in the face of the leflunomide's blockade of the de novo pathway. Notwithstanding the potential benefits of administration of exogenous uridine, therapeutic use of uridine is complicated by its poor bioavailability (about 8%-10%), requiring high dose administration for effective therapy. Moreover, high doses of uridine may cause gastrointestinal complications, including diarrhea, which are poorly tolerated in transplant patients dependent on intestinal function for therapeutic drug administration and which may exacerbate the diarrhea already caused by the leflunomide.

The present invention relates to the surprising discovery that the use of orotic acid alleviates the toxicity typically observed with leflunomide administration. Orotic acid (also known as vitamin B₁₃), an intermediate in the uridine synthetic pathway, appears to eliminate the pyrimidine deficiency caused by the malononitrilamides, metabolites (analogues of the active metabolite) of leflunomide, while avoiding the problems associated with uridine administration.

Accordingly, the invention provides methods and compositions useful in alleviating or reducing toxicity associated with leflunomide administration without reducing its bioactivity, e.g., without reducing its immunosuppressive activity. The present invention uses a bioavailable pyrimidine compound to ameloriate the toxic effects (e.g., anemia, diarrhea, heptotoxicity) caused by leflunomide compounds. As a result, high doses of leflunomide compounds can be administered with minimal danger of toxicity, all the while maintaining the therapeutic efficacy of the leflunomide compound. Co-administration of a leflunomide compound with orally bioavailable pyrimidines, such as orotic acid, provides for treatment opportunities using leflunomide compounds previously believed to be toxic, e.g., the present invention provides methods of reducing the toxicity of A77 1726 (a metabolite of leflunomide) analogs (described hereinbelow) by co-administering a leflunomide compound and, e.g., orotic acid. In addition to orotic acid, it is contemplated that additional analogs and metabolites of orotic acid or other bioavailable pyrimidine compounds may be suitable.

In one aspect, the invention provides pharmaceutical compositions particularly for oral administration. Such pharmaceutical compositions suitably include a leflunomide compound, a bioavailable, especially an orally bioavailable, pyrimidine compound or a salt thereof, and a pharmaceutically acceptable carrier.

In another aspect, the invention provides a method of extending the dosage range of a leflunomide compound. The method involves co-administering to a subject, e.g., a mammal, an effective dose of a leflunomide compound and an orally bioavailable pyrimidine compound or salt thereof, e.g., orotic acid. Thus, the invention provides a method of administering high doses of a leflunomide compound without developing, i.e., reducing, toxicity resulting from leflunomide administration, which method comprises administering to a mammal, e.g., a human, treated with a leflunomide compound an effective amount of a bioavailable pyrimidine compound.

The invention further provides methods of prevention or treatment of certain disease states or processes that are suitably treated with a leflunomide compound. Such disease states or conditions include transplant rejection.

The invention will now be described in detail, those skilled in the art will appreciate that such a description of the invention is meant to be exemplary only and should not be viewed as limitative of the full scope thereof.

The following definitions used in the art may be useful in aiding the skilled practitioner in understanding the invention.

“Ameliorating” means observably reducing, alleviating, inhibiting or diminishing any undesirable effect or symptom of a condition or process associated with a disease state or any undesirable effect of a treatment of a disease state. For example, “amelioration of the effects of pyrimidine biosynthesis inhibition” may refer to any observable reduction in side effects caused by pyrimidine biosynthesis inhibition. Suitably, at least a 50% reduction in symptoms or side effects may be observed.

The term “co-administration” includes administration of two or more agents in a single unitary dosage form, administration of agents concurrently, and administration of agents sequentially, as long as they are given in a manner sufficient to allow both agents to achieve effective concentrations in the body. The agents may be in an admixture, as, for example, in a single tablet, or simply given concurrently. The agents may also be administered by different routes, e.g., one agent may be administered intravenously while the second agent is administered orally. In sequential administration, one agent may directly follow administration of the other or the agents may be administered episodically, i.e., one can be given at one time followed by the other at a later time.

An “effective amount” of a compound, as used herein, means that amount of the compound or composition administered to a subject which is effective to produce its intended function, e.g., in one embodiment of the invention, prevention of transplant rejection. Thus, a “therapeutically effective amount” is an amount effective to produce therapeutic results. A “toxicity-reducing effective amount” is an amount effective to reduce toxicity. Typically, administration of effective amounts to a subject results in observable amelioration of undesirable effects or symptoms of the condition or disease process which the subject is being treated.

“Extending a dosage range” refers to providing a means by which greater doses of an agent may be administered to a subject to increase therapeutic effectiveness. Typically, extending a dosage range is useful, e.g., when efficacy of an agent is dose dependent but increased doses of the agent also leads to dose dependent toxicity. Alternatively, the term “extending a dosage range” may refer to administering agents believed to be toxic at any dosage.

A “leflunomide compound” refers generally to leflunomide, its analogs, its metabolites and analogs thereof.

Leflunomide is an isoxazole derivative with a chemical name of N-(4-trifluoromethylphenyl)-5-methylisoxazol-4-carboxamide having the following chemical formula (I):

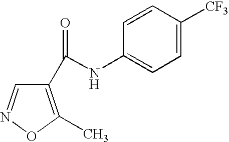

Analogs of leflunomide which may be useful in the practice of the methods of the invention may be represented by formula (II): 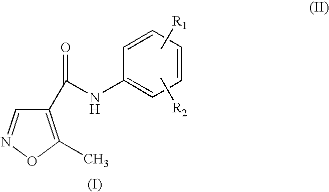
wherein R₁ and R₂ are independently selected from the group consisting of —CF₃, —H, —Cl, —F, —Br, —CN, —COOH, —OCH₃, —NH—CO—CH₂Cl and NH—CO—CH₂Br. (See, e.g., U.S. Pat. Nos. 4,087,535; 6,133,301; and 6,727,272)

Leflunomide's active metabolite is referred to as “A77 1726” (2 cyano-3hydroxy-N-(4-trifluromethylphenyl)-buteneamide). After administration, leflunomide is rapidly converted to its active open-ring form, A77 1726, and is shown herein as formula (III): 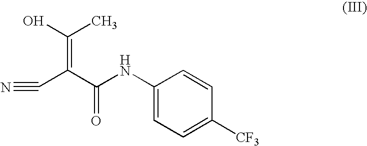
This compound, a member of the malononitrilamide class of compounds, appears to account for leflunomide's activity and toxicity. Although its mechanism of action is not completely understood and wishing not be bound of any particular theory, A77 1726 is believed to exhibit at least two biochemical activities in vivo: inhibition of dihydroorotic acid dehydrogenase (DHODH) in the de novo synthesis of pyrimidine nucleotide triphosphates; and inhibition of selected tyrosine kinases involved in T-cell, B-cell, vascular smooth muscle cell, endothelial cell, fibroblast and tumor cell signaling cascades. A77 1726 also has been reported to block NFkB and AP-1 activation in peripheral blood lymphocytes in vitro. Additional mechanisms remain to be discovered.

Suitable malononitrilamide compounds which are analogs of A77 1726 may be useful in the practice of the methods of the invention and may be represented by formula (IV): 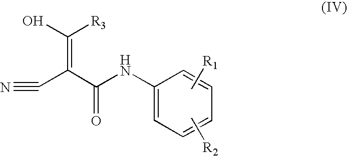
wherein R₁ and R₂ are independently selected from the group consisting of —CF₃, —H, —Cl, —F, —Br, —CN, —COOH, —OCH₃, —NH—CO—CH₂Cl and NH—CO—CH₂Br and wherein R₃ is selected from the group consisting of C₁₋₅ alkyl, C₂₋₅ alkenyl, C₂₋₅ alkynyl, and C₃₋₆ cycloalkyl. Compounds of formula (IV) include FK7778 and FK779 wherein R₁ is —H, R₂ is —CF₃ and R₃ is butynyl (i.e., 2-cyano-3-hydroxy-N-[4-(lufluaromethyl) phenyl]-2-hepten-6-ynoic acid amide) and R₁ is —H, R₂ is cyano and R₃ is cyclopropyl (i.e., 2-cyano-3-hydroxy-3-cyclopropyl-N-(4-cyanophenyl)-propenic acid amide), respectively.

In some embodiments of the methods of the invention, the leflunomide compound is administered as a prodrug to subjects and subsequently converted in vivo to its active malononitrilamide compound, defined above. It is contemplated, however, that the malononitrilamide compound may also be directly administered, and the term “leflunomide compound”, as defined above, also refers to malononitrilamide compounds. It is to be understood that discussion herein regarding leflunomide compound administration is meant to be inclusive of malononitrilamide compound administration, as appropriate.

Leflunomide and its analogs can be prepared by known methods such as those described in U.S. Pat. No. 6,723,855; U.S. Pat. No. 6,727,272; U.S. Pat. No. 6,133,301; U.S. Pat. No. 5,905,090; U.S. Pat. No. 4,087,535; U.S. Pat. No. 4,351,841; and U.S. Pat. No. 4,965,276, all of which are incorporated herein by reference in their entireties. Leflunomide is also commercially available from chemical suppliers, such as SynQuest Corp. (Chicago, Ill.).

As used herein, “bioavailable” in reference to a pyrimidine compound is one that is at least about 20% bioavailable after administration. “Orally bioavailable” in reference to a pyrimidine compound is a compound that is at least about 20% bioavailable after oral administration.

The phrase “pharmaceutically acceptable carrier,” as used herein, means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

As used herein, “preventing,” “reducing risk” or “reduced risk” as it applies to a particular condition or disease process, refers to observable results which tend to demonstrate that a particular treatment or treatment regimen has resulted in a significant decrease in incidence of the condition or disease process in a treated population, as compared to an untreated or control population. Suitably, risk is reduced, or a condition is prevented, if at least 50% of the treated population are not afflicted.

As used herein, a “pyrimidine compound” refers to a compound that is bioavailable, especially orally bioavailable, and useful either directly or as intermediates in pathways for supplying pyrimidine nucleotides. A suitable pyrimidine compound is, e.g., orotic acid. Other suitable pyrimidine compounds include orotic acid salts, triacetyl uridine and salts thereof, cytidine, acylated cytidine and salts thereof.

It is to be understood that the phrase “a salt thereof,” when used herein to refer to pharmaceutical compositions, means physiologically compatible salts which are pharmaceutically acceptable. Examples of suitable salts are alkali metal (e.g., sodium), alkaline earth metal (e.g., calcium, magnesium) and ammonium salts, including those of physiologically tolerated organic ammonium bases.

As used herein, the term “treating” means observably reducing any undesirable effect or symptom of a condition or process associated with a disease state or any undesirable effect of a treatment of a disease state. Suitably, at least a 50% reduction in symptoms or side effects may be observed in a treated subject.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. These are only examples of what is specifically intended.

In one embodiment, the present invention provides an effective method for reducing the risk of toxicity of leflunomide compounds used for the treatment of transplant rejection. Particularly, the present invention relates to therapeutic methods for ameliorating the risk of toxic side effects of a leflunomide compound, and thus permitting extending dosing of such compounds. The present invention provides treatment of a patient suffering from the toxic side effects of a leflunomide compound with an orally bioavailable pyrimidine compound based on a novel treatment protocol. The pyrimidine compound is suitably orotic acid, a salt thereof (e.g., sodium orotate), or a triacetyluridine. The pyrimidine compound is provided to the patient to significantly reduce the toxic effects of a leflunomide compound, e.g., anemia and diarrhea resulting in reduced hematocrit and weight loss. These attributes are achieved through specific properties of the pyrimidine compounds and the novel treatment protocol as described herein.

A suitable pyrimidine compound is orotic acid. Orotic acid is found in small concentrations in the blood of healthy individuals. Elevated levels appear to be free of any appreciable complications in humans and animals. Several conditions are known, however, in which orotic acid levels in the blood are elevated, e.g., in urea synthesis defects, in individual Hereditary Oroticaciduria treated with uridine for years, and in patients receiving allopurinol, without recognized specific damage. In addition, it is recognized that blood levels of orotic acid are elevated several fold in renal failure without specifically recognized toxicity.

Orotic acid may be prepared by condensation of urea with the monoethyl ester of oxalacetic acid in methanol. Other preparation methods, including those utilizing biotechnological methods known in the art, are also suitable. Orotic acid may be administered in its free acid form, or may be administered as a pharmaceutically acceptable salt. Examples of suitable salts are alkali metal (e.g., sodium orotate), alkaline earth metal (e.g., magnesium orotate or calcium orotate) and ammonium salts, including those of physiologically tolerated organic ammonium bases. Orotic acid is also commercially available from chemical suppliers, such as Aldrich (Milwaukee, Wis.).

Also included among the bioavailable pyrimidine compounds of the invention are those comprising certain known acyl derivatives of uridine, i.e., acylated uridines, e.g., 2′, 3′, 5′-tri-O-acetyl uridine (or triacetyluridine (TAU)), 2′, 3′, 5′-tri-O-propionyl uridine, or 2′, 3′, 5′-tri-O-butyryl uridine. TAU, for example, is orally bioavailable. TAU and other acyl derivatives of uridine can be made by methods known in the art (see, e.g., U.S. Pat. No. 6,316,426; U.S. Published Patent Application 2002/0035086 and references cited therein, all of which are incorporated herein by reference); TAU is also commercially available through SP-Chemicals, Ludwigshofen, DK.

The pyrimidine compounds of the invention also include cytidine and certain acyl derivatives of cytidine, i.e., acylated cytidines, e.g., 2′, 3′, 5′-tri-O-acetyl cytidine (or triacetylcytidine or TAC), 2′, 3′, 5′-tri-O-propionyl cytidine, or 2′, 3′, 5′-tri-C-butyryl cytidine. TAC and other acyl derivatives of cytidine can be made by methods known in the art (see, e.g., U.S. Published Patent Application 2002/0035086 and references cited therein, all of which are incorporated herein by reference).

Suitably, the pyrimidine compound may be administered in an amount that is approximately that which is needed to provide the daily pyrimidine synthesis requirements minus what is provided through the salvage pathway. The total pyrimidine synthesis in adult humans is estimated to be from about 4 mmol/day to about 12 mmol/day, or about 450 to about 700 mg of uridine per day. (Bono V H, Weissman S M, Frei E. The effect of 6-azauridine administration on de novo pyrimidine production in chronic myelogenous leukemia. J Clin Invest 1964; 43:1486; Smith L H Jr. Pyrimidine Metabolism in Man. New Engl J of Med 1973; 288:764-772.) For orotic acid, this would amount to approximately 1000 mg per day. It is not believed to be necessary, however, to provide the entire daily supply of pyrimidine since the salvage pathway provides some of the total. It is believed that the bioavailability of orotic acid is approximately 50%. Therefore, for oral administration in an adult, an effective amount of orotic acid would be about 500 mg to about 2,000 mg per day. A similar dosing is contemplated for TAU.

For patients being treated with a leflunomide compound, the targeted blood level of active metabolite (A77 1726) is suitably between about 50 μg/mL and about 100 μg/mL. The maintenance dose may be adjusted by one of ordinary skill in the art to attain the desired blood level range of active metabolite. If pyrimidine deficiency is prevented with co-administration of a pyrimidine compound, the targeted blood level of active metabolite may be substantially higher, e.g. about 200 μg/mL or about 600 μM.

Mammalian transplant recipients, such as kidney recipients and bone marrow recipients, may be suitably treated in accordance with the present invention. Typically, a human transplant recipient is administered a leflunomide compound at a dose of about 100 mg per day for five days, and then 40 mg per day thereafter as a maintenance dose. The co-administration of the leflunomide compound and a pyrimidine compound will extend the therapeutic dose of the leflunomide compound to more than 200 mg/patient/day. This method will prevent the development of or reduce the risk of toxicity (e.g., anemia, diarrhea, hepatotoxicity) and will result in achieving concentrations of the leflunomide compounds that can suppress rejection. It is expected that the use of this methodology will allow up to 10-fold or higher increase in dosage level of leflunomide compounds with minimal danger of developing toxicity to the patient. In other words, the present invention provides a method of administering a toxic dose of a leflunomide compound by administering an effective amount of a pyrimidine compound. By “toxic dose” or “high dose” is meant a dose of the leflunomide compound which when administered to a mammal such as a human often results in the toxic effects, e.g., anemic and diarrhea as well as other pathological changes. In humans, a high dose may be more than 200 mg per day.

In administration of leflunomide compounds, toxicity-reducing effective amounts of the bioavailable pyrimidine compounds are co-administered to subjects with allografts or xenografts, thereby ameloriating the toxic effects of the leflunomide compounds, i.e., weight gain is promoted and hematocrit maintained, with significantly less risk of toxicity than is observed after the same amount of leflunomide compound alone is administered. The risk of toxicity, associated with the administration of high doses of leflunomide compounds, is lowered by co-administering the leflunomide with a pyrimidine compound, especially an orally bioavailable pyrimidine compound. Thus, the combination therapy for use in accordance with the present invention provides an improved therapeutic index relative to leflunomide compounds alone given in conventional protocols. The treatment protocol in accordance with the present invention provides reduced risk of toxicity, (e.g., improved weight gain and hematocrit) i.e., little or no clinical symptoms or signs of toxicity.

The pyrimidine compounds of the present invention given in the illustrated dosing regimen, thus, overcome the toxicities of leflunomide compounds and can be considered beneficial agents for the control and treatment of toxicity associated with treatment with leflunomide compounds. In such combination therapy, the leflunomide compound may be co-administered with the pyrimidine compound concurrently, sequentially, or in a unitary formulation. For efficiency, ease of administration and patient compliance, the latter is especially suitable.

A pharmaceutical composition of a leflunomide compound and a bioavailable pyrimidine compound is suitably formulated in unit dosage form of about 500 mg to about 2000 mg of pyrimidine compound and about 20 mg to about 100 mg of leflunomide compound. Lower doses of pyrimidine compound may be adequate for children or individuals with reduced clearance of pyrimidines, such as individuals with reduced kidney function or other conditions that might reduce pyrimidine elimination.

The dosage form of compositions of the invention is not particularly limited, and any form suitable for oral administration may be used in accordance with standard formulation procedures known in the art. Examples of dosage forms suitable for oral administration include, but are not limited to, solid formulations and aqueous formulations. Solid formulations suitable for oral administration include capsules, tablets, powders or granules, and may include excipients such as lactose, glucose, sucrose or mannitol; a disintegrator such as starch or sodium alginate; a lubricant such as magnesium stearate or talc; a binder such as polyvinyl alcohol, hydroxypropylcellulose or gelatin; a surfactant such as fatty acid ester; and a plasticizer such as glycerine, and the like. Aqueous formulations suitable for oral administration include solutions, emulsions, syrups and suspensions. Such formulations may also include sugars such as sucrose, sorbitol or fructose; glycols such as polyethylene glycol or propylene glycol, oils such as sesame oil, olive oil or soybean oil, antiseptics such as p-hydroxybenzoate, and flavors such as strawberry and peppermint.

While, perhaps, less convenient than an oral formulation, it is also contemplated that the compositions may be formulated for rectal administration in accordance with standard formulations procedures known in the art. Examples of dosage forms suitable for rectal administration include solid suppositories, mucoadhesive suppositories, solutions, suspensions, retention enemas, gels, forms and ointments.

It is further contemplated that a dosage form of the compositions in accordance with the present invention may be formulated for immediate release, delayed release or controlled release. Many controlled release systems are known in the art (see e.g., U.S. Pat. No. 5,529,991). Sustained, controlled or directed release compositions can be formulated, e.g., in liposomes, via laser originated openings or those wherein the active compound is protected with differentially degradable coatings, such as by microencapsulation, multiple coatings, etc.

For example, in diffusional systems, the release rate of drugs is affected by their rate of diffusion through a water-insoluble polymer. There are generally two types of diffusional systems, formulations in which a core of drug is surrounded by polymeric membrane; and matrix devices in which dissolved or dispersed drug is distributed substantially uniformly and throughout an inert polymeric matrix. In actual practice, many systems that utilize diffusion can also rely to some extent on dissolution to determine the release rate.

Common materials used as the membrane barrier coat, alone or in combination, include but are not limited to, hardened gelatin, methyl and ethyl-cellulose, polyhydroxymethacrylare, polyvinylacetate, and various waxes.

In matrix systems, three major types of material are frequently used in the preparation of the matrix systems which include insoluble plastics, hydrophilic polymers, and fatty compounds. Plastic matrices which have been employed include methyl acrylate-methyl methacrylate, polyvinyl chloride and polyethylene. Hydrophilic polymers include methyl cellulose, hydroxypropylcellulose, hydroxpropyl-ethylcellulose, and its derivatives and sodium carboxy-methylcellulose. Fatty compounds include various waxes such as carnauba wax, and glyceryl tristearate. These matrix systems are prepared by methods well known to those skilled in the art. These methods of preparation generally comprise mixing the drug with the matrix material and compressing the mixture into a suitable pharmaceutical layer. With wax matrices, the drug is generally dispersed in molten wax, which is then congealed, granulated and compressed into cores.

The most common method of microencapsulation is coacervation, which involves addition of a hydrophilic substance to a colloidal dispersion. The hydrophilic substance, which operates as the coating material, is selected from a wide variety of natural and synthetic polymers including shellacs, waxes, starches, cellulose acetates, phthalate or butyrate, polyvinyl-pyrrolidone, and polyvinyl chloride. After the coating material dissolves, the drug inside the microencapsule is immediately available for dissolution and absorption. Drug release, therefore, can be controlled by adjusting the thickness and dissolution rate of the coat. For example, the thickness can be varied from less than one μm to 200 μm by changing the amount of coating material from about 3 to 30 percent by weight of the total weight. By employing different thicknesses, typically three of four, the active agent will be released at different, predetermined times to afford a delayed release effect.

Approaches to further reducing the dissolution rate include, for example, coating the drug with a slowly dissolving material, or incorporating the drug into a formulation with a slowly dissolving carrier. Thus, encapsulated dissolution systems are prepared either by coating particles or granules of drug with varying thickness or slowly soluble polymers or by microencapsulation.

While it is contemplated that a unitary oral formulation containing both a leflunomide compound and a bioavailable pyrimidine compound provides ease of administration and patient compliance, it is also understood that the compounds may be administered separately but packaged together, e.g., in a blister pack, with instructions for administration.

Although examples of suitable dosage ranges are provided, it will be appreciated that the specific dosages administered in any given case will be adjusted in accordance with the specific compounds being administered, the disease to be treated, the condition of the subject and other relevant medical factors that may modify the activity of leflunomide, the response of the subject or the amount of bioavailable pyrimidine compound needed, as is well known by those skilled in the art. For example, the specific dose for a particular patient depends on age, body weight, general state of health, diet, the timing and mode of administration, the rate of excretion, and medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol.

The following examples are provide to assist in a further understanding of the invention. The particular materials and conditions employed are intended to be further illustrative of the invention and are not limiting upon the reasonable scope thereof.

### EXAMPLE 1

### Effect of Orotic Acid Administration on Efficacy of Leflunomide in the Treatment of Acute Rejection

Lewis Rats which received heart transplants from Brown-Norway rats were observed for graft survival and inflammation (scored on a 0-3 scale, with 0 being no inflammation). Treatments included 0, 5, 10 or 15 mg/kg of leflunomide in combination with 0 or 100 mg/kg orotic acid. The results are tabulated below.

| | Dose | Dose orotic | Graft | Inflammation |
|---|---|---|---|---|
| Number | leflunomide | acid | survival | score(Mean) |
| animals | (mg/kg/day) | (mg/kg/day) | (days) | (0-3) |
| 8 | 0 | 0 | 6.9 | 3.0 |
| 8 | 5 | 0 | >30 | 2.0 |
| 5 | 5 | 100 | >30 | 2.3 |
| 5 | 10 | 0 | >30 | 1.7 |
| 5 | 10 | 100 | >30 | 1.8 |
| 5 | 15 | 0 | >30 | 1.5 |
| 5 | 15 | 100 | >30 | 1.6 |

Administration of leflunomide reduced the intensity of the rejection reaction, as shown by the inflammation score, in a dose-related fashion. Orotic acid did not significantly affect the efficacy of leflunomide to reduce the intensity of the rejection reaction.

### EXAMPLE 2

### Effect of Orotic Acid on Leflunomide Toxicity as Measured by Changes in Body Weight

As noted previously, the most observed symptoms of experimental leflunomide-induced toxicity are anemia and diarrhea resulting in weight loss or reduced weight gain. Lewis rats with either an allograft or xenograft weighing between 200 and 235 grams were divided into four treatment groups. Each group received 30 mg/kg/day of leflunomide, a high, toxic dose: Group I received leflunomide only; Group II received leflunomide plus 36 mg/kg/day of sodium orotate by gavage; Group III received leflunomide plus 100 mg/kg/day of orotic acid by gavage; and Group IV received 250 mg/kg/day of uridine by IP injection. Weight of each rat was measured at week 1 and week 4 post commencement of therapy. The results are tabulated below.

| | | | |
|---|---|---|---|
| Treatment-dosage | Weight at Week 1 | Weight at Week 4 | Change in body |
| (mg/kg/day) | Grams | grams | weight |

GROUP I (leflunomide only)

| | | | |
|---|---|---|---|
| L30 | 234 | 246 | 13 |
| L30 | 211 | 236 | 25 |
| L30 | 241 | 237 | −4 |
| L 30 | 253 | 264 | 11 |
| L 30 | 246 | 255 | 9 |
| L 30 | 203 | 253 | 50 |
| | | | Mean: 20.8 |

GROUP II

| | | | |
|---|---|---|---|
| L30 + O36 | 270 | 266 | −4 |
| L30 + O36 | 244 | 235 | −9 |
| L30 + O36 | 250 | 218 | −32 |
| L30 + O36 | 205 | 267 | 62 |
| L30 + O36 | 198 | 240 | 42 |
| L30 + O36 | 211 | 262 | 51 |
| | | | Mean: 18.3 |

GROUP III

| | | | |
|---|---|---|---|
| L30 + O100 | 210 | 250 | 40 |
| L30 + O100 | 208 | 265 | 57 |
| L30 + O100 | 209 | 250 | 41 |
| L30 + O100 | 207 | 271 | 66 |
| L30 + O100 | 210 | 268 | 58 |
| L30 + O100 | 197 | 258 | 61 |
| | | | Mean: 53.7 |

GROUP IV

| | | | |
|---|---|---|---|
| L30 + U250 | 207 | 231 | 24 |
| L30 + U250 | 204 | 242 | 38 |
| L30 + U250 | 208 | 230 | 22 |
| L30 + U250 | 197 | 241 | 44 |
| L30 + U250 | 204 | 272 | 68 |
| L30 + U250 | 211 | 275 | 64 |
| | | | Mean: 43.3 |

* L30 refers to administration of 30 mg/kg per day of leflunomide; O36 refers to 36 mg/kg per day of orotic acid; O100 refers to 100 mg/kg per day of orotic acid; and U250 refers to 250 mg/kg per day of uridine given IP.

The results showed that the use of a combination of leflunomide and orotic acid or a salt thereof significantly improved weight gain compared to use of leflunomide alone.

### EXAMPLE 3

### Effect of Orotic Acid on Leflunomide Toxicity as Measured by Hematocrit

The experiment of Example 2 was repeated in Lewis rats and the hematocrit measured weekly for four weeks. The rats receiving treatment were divided into five groups wherein each group received 30/mg/kg/day leflunomide, a toxic, high dose. Group I received leflunomide only. Group II received the leflunomide dose plus 36/mg/kg/day of sodium orotate; group III received the leflunomide dose plus 100 mg/kg/day of sodium orotate; group IV received the leflunomide dose plus 88 mg/kg/day of orotic acid; and group V received the leflunomide dose plus 250 mg/kg/day of uridine given IP. A baseline hematocrit was measured, and hematocrits of each rat were measured at weeks 1-4 post commencement of therapy. The results are tabulated below.

| | | | | | |
|---|---|---|---|---|---|
| Treatment- | | | | | |
| dosage | Hct | Hct | Hct | Hct | Hct |
| (mg/kg/day) | Week 0 | Week 1 | Week 2 | Week 3 | Week 4 |

GROUP I

| | | | | | |
|---|---|---|---|---|---|
| L30 | 51 | 47 | 39 | 31 | 17 |
| L30 | 57 | 52 | 46 | 33 | 20 |
| L 30 | 54 | 55 | 54 | 37 | 30 |
| L 30 | 55 | 49 | 48 | 33 | 24 |
| L 30 | 52 | 50 | 46 | 37 | 22 |
| L 30 | 53 | 53 | 44 | 41 | 30 |
| | | | | | Mean: 23.8 |

GROUP II

| | | | | | |
|---|---|---|---|---|---|
| L30 + O36 | 53 | 50 | 48 | 39 | 20 |
| L30 + O36 | 57 | 50 | 49 | 32 | 14 |
| L30 + O36 | 53 | 51 | 39 | 27 | 10 |
| L30 + O36 | 54 | 52 | 49 | 47 | 44 |
| L30 + O36 | 51 | 53 | 40 | 36 | 35 |
| L30 + O36 | 52 | 49 | 44 | 30 | 20 |
| | | | | | Mean: 23.8 |

GROUP III

| | | | | | |
|---|---|---|---|---|---|
| L30 + O100 | 53 | 48 | 43 | 41 | 32 |
| L30 + O100 | 52 | 49 | 51 | 46 | 44 |
| L30 + O100 | 51 | 46 | 44 | 42 | 45 |
| L30 + O100 | 57 | 53 | 50 | 41 | 33 |
| L30 + O100 | 51 | 49 | 46 | 34 | 22 |
| L30 + O100 | 52 | 51 | 53 | 43 | 46 |
| | | | | | Mean: 37 |

GROUP IV

| | | | | | |
|---|---|---|---|---|---|
| L30 + U250 | 52 | 47 | 48 | 50 | 46 |
| L30 + U250 | 56 | 50 | 51 | 51 | 49 |
| L30 + U250 | 54 | 46 | 43 | 41 | 25 |
| L30 + U250 | 51 | 51 | 48 | 38 | 24 |
| L30 + U250 | 53 | 49 | 54 | 51 | 44 |
| L30 + U250 | 54 | 54 | 49 | 43 | 38 |
| | | | | | Mean: 37.7 |

GROUP V

| | | | | | |
|---|---|---|---|---|---|
| L30 + OA88 | 56 | 50 | 53 | 52 | 50 |
| L30 + OA88 | 54 | 51 | 54 | 52 | 54 |
| L30 + OA88 | 52 | 49 | 44 | 38 | 32 |
| | | | | | Mean: 45.3 |

The results demonstrated that use of the combination of leflunomide and orotic acid or sodium orotate, provided significantly higher hematocrits than in the use of leflunomide alone.

As used in this specification and the appended claims, the singular forms “a,” “an,” and “the” include plural referents unless the content clearly dictates otherwise. Thus, for example, reference to a composition containing “a pyrimidine compound” includes a mixture of two or more pyrimidine compounds. It should also be noted that the term “or” is generally employed in its sense including “and/or” unless the content clearly dictates otherwise.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

The invention has been described with reference to various specific embodiments and techniques. However, it should be understood that many variations and modifications may be made while remaining within the spirit and scope of the invention.

We claim:

1. A pharmaceutical composition in unit dosage form for oral administration comprising an effective amount of a leflunomide compound; and an orally bioavailable pyrimidine compound, salts thereof or a combination thereof; together in a pharmaceutically acceptable carrier.

2. The composition of claim 1 wherein the pyrimidine compound is orotic acid, a salt thereof, triacetyluridine, a salt thereof, cytidine, a salt thereof, an acylated cytidine, a salt thereof, or a combination thereof.

3. The composition of claim 1, wherein the unit dosage contains 500 mg to 2000 mg of pyrimidine compound.

4. The composition of claim 1, wherein the leflunomide compound is leflunomide, A771726 or FK778.

5. The composition of claim 1, wherein the composition is formulated for controlled release.

6. The composition of claim 1, wherein the composition is formulated for rectal administration.

7. A pharmaceutical composition comprising a formulation for oral administration, the formulation comprising a therapeutically effective amount of leflunomide, and orotic acid or a salt thereof, and a pharmaceutically acceptable carrier.

8. A method of reducing toxicity associated with administration of a leflunomide compound to a patient in need thereof, comprising administering to the patient a toxicity-reducing amount of a bioavailable pyrimidine compound.

9. The method of claim 8, wherein the pyrimidine compound is orotic acid, a salt thereof, triacetyaluridine, a salt thereof, cytidine, a salt thereof, an acylated cytidine, a salt thereof, or a combination thereof.

10. The method of claim 8, wherein the pyrimidine compound is administered orally.

11. The method of claim 8, wherein the pyrimidine compound is administered in a daily dosage of from about 500 mg to about 2000 mg.

12. The method of claim 8, wherein the pyrimidine compound is co-administered substantially simultaneously with the leflunomide compound.

13. The method of claim 8, wherein the patient is a recipient of a transplant.

14. The method of claim 13, wherein the transplant is an allograft or a xenograft.

15. The method of claim 13, wherein the transplant is a heart, a kidney or bone marrow.

16. The method of claim 8, wherein the leflunomide compound is selected from a compound having
a) formula (II): 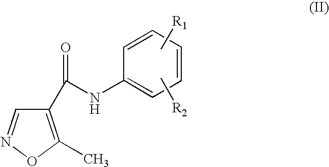
wherein R₁ and R₂ are independently selected from the group consisting of —CF₃, —H, —Cl, —F, —Br, —CN, —COOH, —OCH₃, —NH—CO—CH₂Cl and —NH—CO—CH₂Br;
or formula (IV): 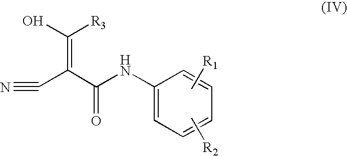
wherein R₁ and R₂ are independently selected from the group consisting of —CF₃, —H, —Cl, —F, —Br, —CN, —COOH, —OCH₃, —NH—CO—CH₂Cl and —NH—CO—CH₂Br, and R3 is selected from the group consisting of C₁₋₅ alkyl, C₂₋₅ alkenyl, C₂₋₅ alkynyl, and C₃₋₆ cycloalkyl.

17. A method of extending the dosage range of a leflunomide compound comprising co-administering to a subject:
a) an effective amount of a leflunomide compound of formula (II): 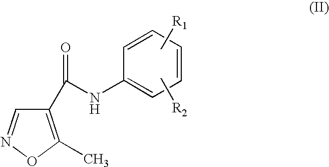
wherein R₁ and R₂ are independently selected from the group consisting of —CF₃, —H, —Cl, —F, —Br, —CN, —COOH, —OCH₃, —NH—CO—CH₂Cl and —NH—CO—CH₂Br;
or formula (IV): 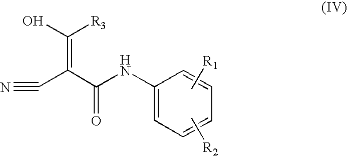
wherein R₁ and R₂ are independently selected from the group consisting of —CF₃, —H, —Cl, —F, —Br, —CN, —COOH, —OCH₃, —NH—CO—CH₂Cl and —NH—CO—CH₂Br; and R₃ is selected from the group consisting of C₁₋₅ alkyl, C₂₋₅ alkenyl, C₂₋₅ alkynyl, and C₃₋₆ cycloalkyl; and
b) a toxicity-reducing amount of an orally bioavailable pyrimidine compound selected from the group consisting of orotic acid, a salt thereof, triacetyl uridine, a salt thereo, cytidine, a salt thereof, an acylated cytidine, a salt thereof, and a combination thereof.

18. A method of administering a toxic dose of a leflunomide compound to a mammal, comprising administering to the mammal an amount of an orally bioavailable pyrimidine compound sufficient to reduce the toxic effects of the leflunomide compound.

19. A method of reducing toxicity associated with the administration of a therapeutically effective amount of a leflunomide compound to a mammal, comprising: orally administering to the mammal a bioavailable pyrimidine compound selected from orotic acid, a salt thereof, triacetyluridine, a salt thereof, cytidine, a salt thereof, an acylated cytidine, a salt thereof, and a combination thereof, in an amount effective to reduce the toxicity without blocking therapeutic 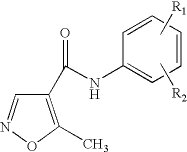
effect of the leflunomide compound, wherein the leflunomide compound is a compound of formula (II) or formula (IV).

20. The method of claim 19 wherein the pyrimidine compound is orotic acid or a salt thereof.

21. A method of treating rejection in a transplant recipient comprising co-administering 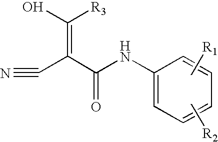
a therapeutically effective amount of a leflunomide compound and a toxicity-reducing effective amount of bio-available pyrimidine compound.

22. The method of claim 21, wherein the pyrimidine compound is orally bio-available.

23. The method of claim 22, wherein the pyrimidine compound is orotic acid, a salt thereof, triacetyl uridine, a salt thereof, cytidine, a salt thereof, an acylated cytidine, a salt thereof, or a combination thereof.

24. A method of achieving an effect in a patient comprising co-administering an effective amount of a leflunomide compound and an effective amount of orotic acid, a salt thereof, triacetyl uridine, a salt thereof, or a combination thereof, wherein the effect is treatment of rejection of a transplant, wherein the transplant is heart, kidney or bone marrow.

25. A pharmaceutical combination comprising a packaging having a plurality containers, at least one container containing a leflunomide compound, at least one other container containing a bioavailable pyrimidine compound, and an instructions for co-administering the leflunomide compound and the pyrimidine compound to a subject who is a transplant recipient.
